(19) United States
(12) Patent Application Publication
(10) Pub. No.: US 2012/0022326 A1
(43) Pub. Date: Jan. 26, 2012

(54) INTUBATING STYLETS AND SYSTEMS AND METHODS FOR INTUBATION

(75) Inventor: Francisco Jaime, Albuquerque, NM (US)

(73) Assignee: STC.UNM, Albuquerque, NM (US)

(21) Appl. No.: 13/131,531

(22) PCT Filed: Dec. 1, 2009
(86) PCT No.: PCT/US09/66293

§ 371 Date: Oct. 6, 2011

Related U.S. Application Data
(60) Provisional application No. 61/119,028, filed on Dec. 1, 2008.

(51) Int. Cl. A61M 16/04; A61B 1/04

(52) U.S. Cl. 600/109; 128/207.14

(57) ABSTRACT

An intubating stylet has a proximal end and a distal end. The intubating stylet includes a housing at the proximal end of the stylet and a tip portion at the distal end of the stylet. The tip portion has a distal gear at its proximal end. An outer shaft extends from the housing to the tip portion and contains an inner shaft defining a hollow lumen. A distal end of the inner shaft includes a proximal gear coupled with the distal gear of the tip portion such that rotation of the inner shaft causes articulation of the tip portion. The stylet further includes a drive member in the housing and a control member coupled with the drive member. The drive member is coupled to the inner shaft and configured to rotate the inner shaft, and the control member is configured to receive a user input and to translate the input into operation of the drive member.

### CROSS-REFERENCE TO RELATED APPLICATIONS

This application is related to U.S. provisional patent application No. 61/119,028, entitled “TOPICALIZATION CATHETERS, INTUBATING STYLETS, AND SYSTEMS FOR INTUBATION,” filed on Dec. 1, 2008, the content of which is incorporated herein by reference in its entirety.

### TECHNICAL FIELD

The present disclosure relates to intubating stylets and systems and methods for intubation. More particularly, the disclosure relates to articulating intubating stylets, systems for controlling movement and articulation of intubating stylets, and methods for intubation using articulating intubating stylets.

### BACKGROUND

One of the most important roles of the anaesthesiologist is maintaining the patient's airway open and patent during the delivery of general anaesthesia. An endotracheal tube is used in most cases of general anaesthesia. The inability to intubate the patient's trachea with an endotracheal tube is one of the main reasons for malpractice lawsuits against anaesthesiologists in the United States. Unanticipated difficult airways are the cause of most failed intubations. Inability to intubate and ventilate the patient can lead to death. Having the proper equipment immediately available on a routine basis during all intubations will help to prevent this situation.

It may be desirable to provide an affordable gear-driven intubating stylet which may be made immediately available for every intubation wherever it may occur: hospital, operating room, surgery center, out patient clinic, accident scene, battlefield, etc.

### SUMMARY

According to some aspects, an intubating stylet has a proximal end and a distal end. The intubating stylet includes a housing at the proximal end of the stylet and a tip portion at the distal end of the stylet. The tip portion has a distal gear at its proximal end. An outer shaft extends from the housing to the tip portion and contains an inner shaft defining a hollow lumen. A distal end of the inner shaft includes a proximal gear coupled with the distal gear of the tip portion such that rotation of the inner shaft causes articulation of the tip portion. The stylet further includes a drive member in the housing and a control member coupled with the drive member. The drive member is coupled to the inner shaft and configured to rotate the inner shaft, and the control member is configured to receive a user input and to translate the input into operation of the drive member.

### BRIEF DESCRIPTION OF THE DRAWINGS

FIG. 1 is a cutaway side view of an exemplary intubating stylet in accordance with various aspects of the disclosure.

FIG. 2 is an enlarged view of the proximal end of the exemplary stylet of FIG. 1.

FIG. 3 is an enlarged view of the distal end of the exemplary stylet of FIG. 1.

FIG. 4 is a cutaway side view of an exemplary telescoping intubating stylet in an extended configuration in accordance with various aspects of the disclosure.

FIG. 5 is a cutaway side view of the exemplary telescoping intubating stylet of FIG. 4 in a retracted configuration in accordance with various aspects of the disclosure.

### DETAILED DESCRIPTION

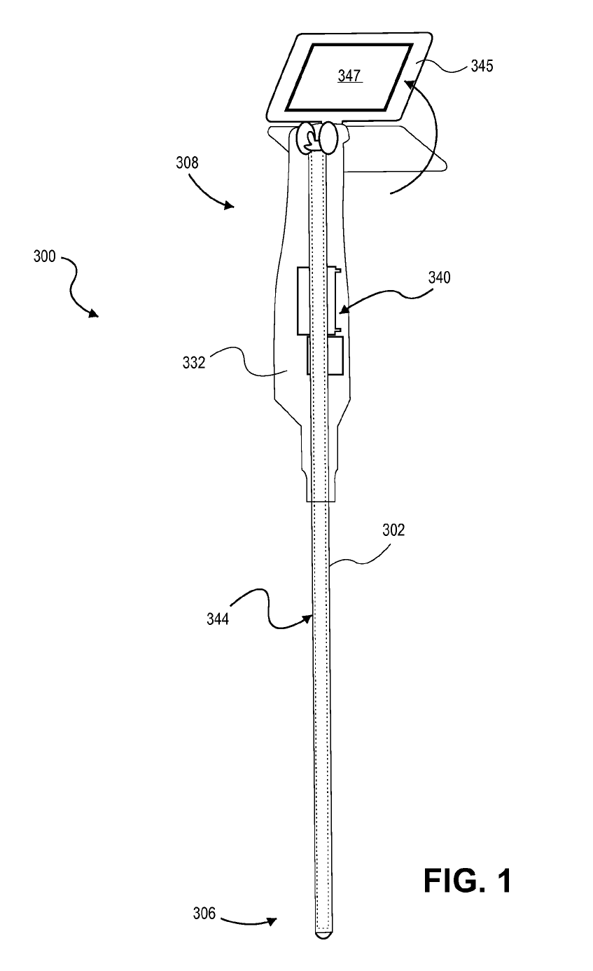

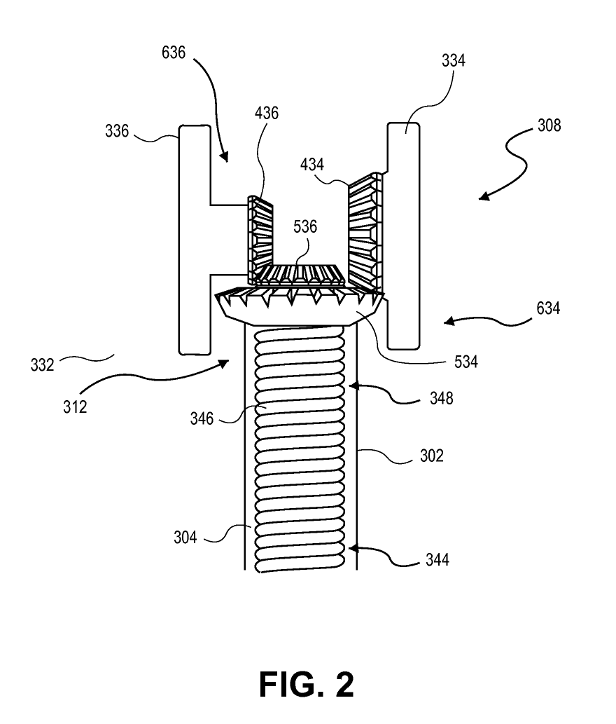

FIG. 1 shows an exemplary articulating intubating stylet 300 in accordance with various aspects of the disclosure. The intubating stylet 300 may comprise an outer shaft 302 defining an internal lumen 304 (FIG. 2). According to some aspects, the outer shaft 302 may be tube-shaped and comprise a non-toxic, flexible plastic material, such as, for example, vinyl, polyethylene, nylon, ethylene-propylene copolymer, plasticized polyvinyl chloride resin, or the like. According to some aspects, the outer shaft 302 of the intubating stylet 300 may have an outside diameter sized to substantially correspond to the inside diameter of an endotracheal tube to be placed over the outer shaft 302 of the stylet 300. That is, the outside diameter should be selected to substantially match the inside diameter of the endotracheal tube such that outer shaft 302 just barely fits inside the endotracheal tube in a slideable manner, as would be understood by those skilled in the art. For example, the outside diameter may range from about ⅛ inch to about ⅜ inch (about 3 mm to about 9 mm) since most conventional endotracheal tubes have an inside diameter between about 3 mm and about 9 mm. In some embodiments, the outside diameter of the outer shaft 302 may be about ¼ inch (about 6 mm). Of course, other size shafts and endotracheal tubes are contemplated by this disclosure.

The stylet 300 may include a distal end 306 and a proximal end 308. According to various aspects, the intubating stylet 300 may have a length similar to conventional intubating stylets, which range, for example, from about 8 inches (about 20 cm) to about 15 inches (about 38 cm). The proximal end 308 of the stylet 300 may include a housing 332 rotatably coupled relative to the outer shaft 302. According to some aspects, the housing 332 may be configured as a handgrip.

As would be understood by persons of ordinary skill in the art, the housing 332 may be detachable from the outer shaft 302 in accordance with known structures and methods. Therefore, if a practitioner realizes that an endotracheal tube disposed on the stylet 300 for insertion into a patient's trachea is not the desired size or length, the practitioner can detach the housing 332 from the outer shaft 302, remove the undesired endotracheal tube from the stylet 300, and place another endotracheal tube on the outer shaft 302 without removing the stylet 300 from a patient's trachea. Extensions to the outer shaft 302 may be added if necessary.

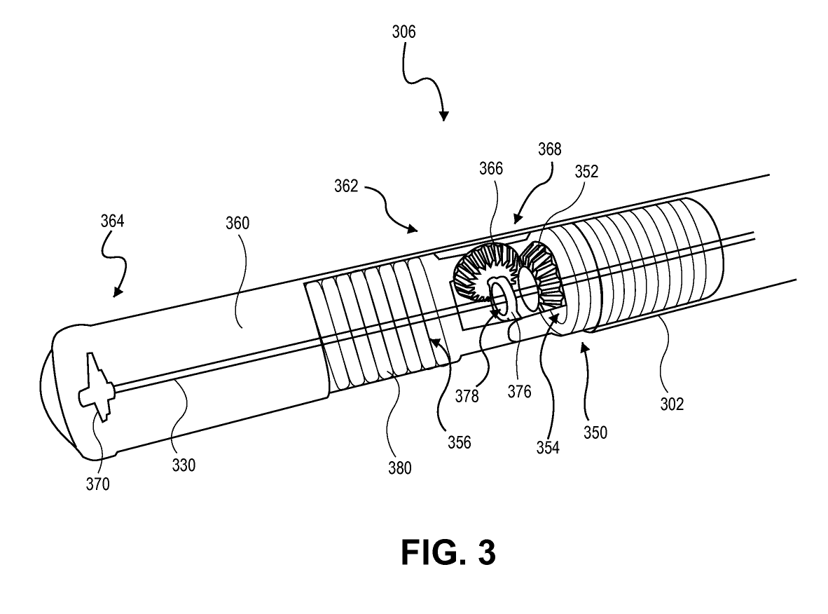

According to various aspects, the stylet 300 may include an inner shaft 344 that extends through the lumen 304 from the proximal end 308 of the stylet 300 to the distal end 306 of the stylet 300. The inner shaft 344 is rotatable relative to the outer shaft 302 and the housing 332. In some aspects, as illustrated in FIG. 2, the inner shaft 344 may comprise a coil spring 346, for example, a metal coil spring. The coil spring 346 should be flexible enough to flex in any direction, but at the same time should be relatively stiff to prevent undesired bending. The coil spring 346 should be tightly wound such that rotation of a proximal end 348 of the coil spring 346 causes a distal end 350 (FIG. 3) of the coil spring 346 to rotate in a similar manner. Meanwhile, rotation of the proximal end 348 of the coil spring 346 should not cause appreciable longitudinal compression of the coil spring 346. For example, this operation of the coil spring may be compared to that of a speedometer cable or a flexible wrench extension such as, for example, that shown in U.S. Pat. No. 5,697,269 to Tseng.

The housing 332 may contain internal electronics 340 such as, for example, a power source, a camera, and/or a light source. The power source, such as, for example, a battery pack, may be configured to power the camera and/or the light source. The camera may comprise, for example, a digital camera. The camera may be electrically coupled with a video monitor 345. The video monitor 345 may be pivotably coupled to the housing 332 at a position superior to the control member 334 such that a display 347 associated with the video monitor 345 is visible to a user during manipulation of the stylet 300. When not in use, the video monitor 345 may be pivoted downward toward the outer shaft 302 for more compact storage.

The stylet 300 may include an optional videoscope and light assembly 330 (FIG. 3) extending through the internal lumen 304 from the proximal end 308 to the distal end 306. The coil spring 346 provides a hollow lumen 354 configured to receive the optional videoscope and light assembly 330. The videoscope and light assembly 330 may be flexible so as to be able to articulate with the outer and inner shafts 302, 344. Of course, in some aspects, the videoscope may include a wireless camera.

Referring now to FIG. 2, the housing 332 may include one or more control members 334, 336 extending therefrom. The control members 334, 336 may comprise, for example, dials 434, 436 having gears 534, 536 associated therewith. The control members 334, 336 are structured and arranged such that rotation of first dial 434 causes like rotation of first gear 534, and rotation of second dial 436 causes like rotation of second gear 536.

A proximal end 312 of the outer shaft 302 includes a third bevel gear 322 associated therewith. The first bevel gear 534 is mechanically coupled to the third bevel gear 322 to form an outer shaft bevel gear assembly 634. The bevel gear assembly 634 controls relative rotation between the outer shaft 302 and the housing 332. That is, rotation of the first dial 434 in a first direction causes clockwise rotation of the outer shaft 302 relative to the housing 332, and rotation of the first dial 434 in a second direction, opposite to the first direction, causes counter-clockwise rotation of the outer shaft 302 relative to the housing 332.

The proximal end 348 of the inner shaft 344 includes a fourth bevel gear 324 associated therewith. The second bevel gear 536 is mechanically coupled to the fourth bevel gear 324 to form an inner shaft bevel gear assembly 636. The bevel gear assembly 636 controls relative rotation between the inner shaft 344 and the outer shaft 302. That is, rotation of the second dial 436 in a first direction causes clockwise rotation of the coil spring 346 relative to the outer shaft 302, and rotation of the second dial 436 in a second direction, opposite to the first direction, causes counter-clockwise rotation of the coil spring 346 relative to the outer shaft 302.

Referring now to FIG. 3, the distal end 306 of the stylet 300 may comprise a tip portion 360 having a proximal end 362 and a distal end 364. According to some aspects, the tip portion 360 may comprise a structure separate from the inner shaft. For example, the tip portion 360 may contain a coil spring 380, for example, a metal coil spring, to facilitate flexibility of the tip portion 360. As illustrated, a distal end 350 of the inner shaft 344 may end short of the tip portion 360. For example, in the case of a 15 inch stylet, the coil spring 346 may extend from the proximal end 308 of the outer shaft 302 through the first 14 inches of the lumen 304.

The distal end 350 of the inner shaft 344 may include a fifth bevel gear 352 associated with the coil spring 346. The tip portion 360 may have a sixth bevel gear 366 fixedly attached at its proximal end 362. An axle 376 may be fixedly coupled with the sixth bevel gear 366 for rotation therewith. The axle 376 include a central lumen 378 for receiving the optional videoscope and light assembly 330 as it extends from the lumen 354 of the coil spring 346 to the tip portion 360.

The fifth bevel gear 352 is mechanically coupled to the sixth bevel gear 366 to form a distal bevel gear assembly 368. The distal bevel gear assembly 368 controls articulation of the tip portion 360 relative to the outer shaft. That is, rotation of the second dial 436 in a first direction causes clockwise rotation of the coil spring 346 relative to the outer shaft 302, which in turn causes rotation of the fifth bevel gear 352, which causes rotation of the sixth bevel gear 366. Since the sixth bevel gear 366 is fixedly attached to the tip portion 360, rotation of the sixth bevel gear 366 causes articulation of the tip portion 360 in a plane substantially perpendicular to a longitudinal axis of the outer shaft 302. Rotation of the second dial 436 in a second direction, opposite to the first direction, causes counter-clockwise rotation of the coil spring 346 relative to the outer shaft 302, which in turn causes rotation of the fifth bevel gear 352, which causes rotation of the sixth bevel gear 366 and articulation of the fixedly coupled tip portion 360 in the same plane substantially perpendicular to a longitudinal axis of the outer shaft 302, but in an opposite direction.

A conduit 356 may extend from the lumen 354 of the coil spring 346 to the distal end 364 of the tip 360 such that the optional videoscope and light assembly 330 may terminate at the distal end 364 with a light and/or camera lens assembly 370, which may allow for direct visualization at the tip portion 360, for example, via the video monitor 345 and display 347.

In operation, movement of the first control member 334 would cause the outer shaft to rotate clockwise or counter-clockwise relative to the housing 332. Movement of the second control member 336 would cause the inner shaft 344 to rotate clockwise or counter-clockwise relative to the outer shaft 302, which would then impart articulation of the tip portion 360 via the distal bevel gear assembly 368. According to various aspects, the tip portion 360 may, for example, be rotated 120 degrees anteriorly and posteriorly from a vertical position. The outer shaft 302 may be rotated 360 degrees. Thus, the combination of the two dials allows for every point on at least a half dome to be accessed by the distal tip of the stylet.

In use, an endotracheal tube (not shown) would be passed over the gear-driven intubating stylet 300. A patient's oropharynx would be opened by conventional techniques using a laryngoscope. The endotracheal tube and stylet 300 will then be routed through the mouth of a patient toward the trachea (as per standard intubation procedure). If the vocal cords are visualized, then the trachea is intubated in a conventional fashion using the stylet 300 as a routine intubating stylet. If the vocal cords are not visualized, then the stylet may be used to visualize the vocal cords. The tip portion 360 may be moved via by the control member 334 and the stylet 300 is then advanced toward the vocal cords. Although the vocal cords may not be seen directly, they will be seen on the display 347 of the video monitor 345. The tip portion 360 may then be advanced through the vocal cords. The endotracheal tube may then be slid distally relative to the stylet 300 and into the trachea. With the patient having been successfully intubated, the stylet 300 may then be removed from the patient's trachea and mouth.

It should be appreciated that the articulating intubating stylet, in some aspects, may comprise the one or more control members configured as one or more joysticks. In such an embodiment, the stylet may include a servomotor electrically coupled with the one or more joysticks and mechanically coupled with the proximal ends of the outer and inner shafts to control relative rotation between and articulation of the outer and inner shafts, respectively.

It should also be appreciated that, according to some aspects, the inner shaft 344 may comprise links similar to a bicycle chain. The links may allow for flexibility along the x-y plane, while providing stiffness when rotating about the y axis. Rotation around the y axis would then be transmitted along the entire length of the inner shaft 344.

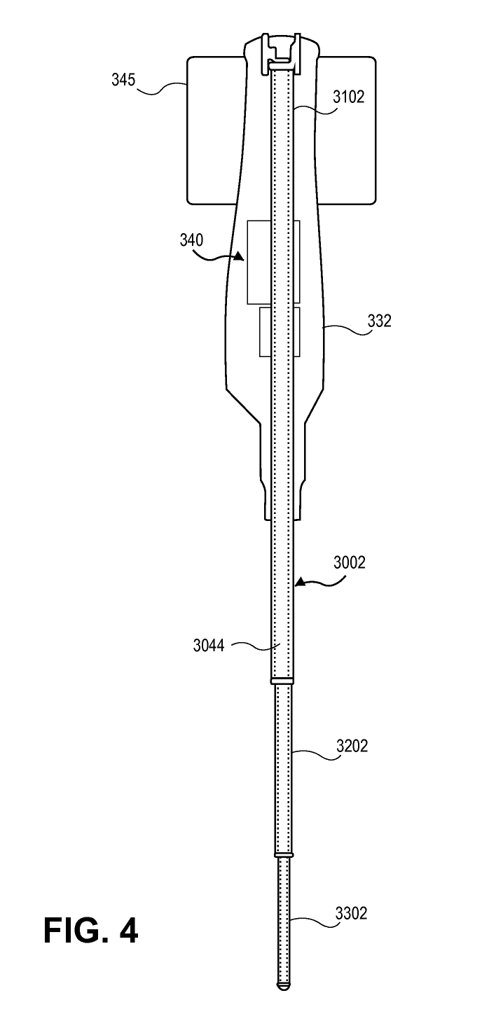

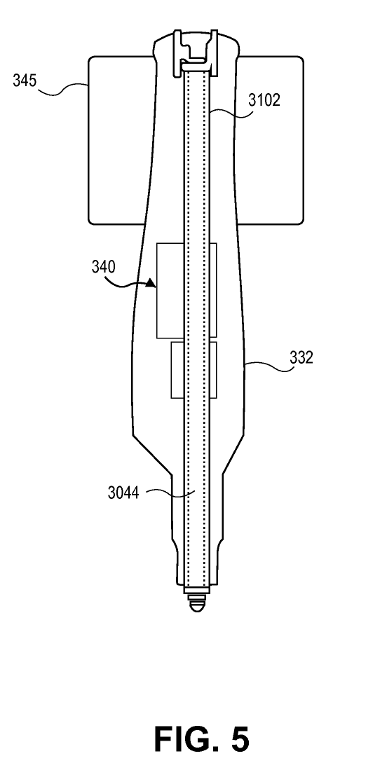

Referring now to FIGS. 4 and 5, an exemplary telescoping articulating intubating stylet 3000 in accordance with various aspects of the disclosure is illustrated. As shown in FIG. 4, the outer shaft 3002 may comprise one or more telescoping members 3102, 3202, 3302. The first telescoping member 3102 may have a coupling (not shown) at its distal end 3108 structured and arranged to engage a complimentary coupling (not shown) at a proximal end 3206 of the second telescoping member 3202. Similarly, the second telescoping member 3202 may have a coupling (not shown) at its distal end 3208 structured and arranged to engage a complimentary coupling (not shown) at a proximal end 3306 of the third telescoping member 3302. Any conventional coupling that would be releasable in a longitudinal direction while being capable of transmitting rotational motion from one telescoping member to another may be employed, including for example, a notch/detent, a spline, or the like. Although three telescoping member 3102, 3202, 3302 are illustrated, it should be appreciated that more or less than three telescoping members may be provided.

Although not shown, it should be appreciated that the inner shaft 3044 may comprise one or more telescoping members (not shown). Similar to the outer shaft 3002, the first telescoping member of the inner shaft 3044 may have a coupling (not shown) at its distal end structured and arranged to engage a complimentary coupling (not shown) at a proximal end of the second telescoping member. Similarly, the second telescoping member may have a coupling (not shown) at its distal end structured and arranged to engage a complimentary coupling (not shown) at a proximal end of the third telescoping member. Any conventional coupling that would be releasable in a longitudinal direction while being capable of transmitting rotational motion from one telescoping member to another may be employed, including for example, a notch/detent, a spline, or the like. Although three telescoping member of the inner shaft are disclosed, it should be appreciated that more or less than three telescoping members may be provided, with the inner shaft and outer shaft typically having the same number of telescoping members.

It should be appreciated that, in some aspects, exemplary stylets may employ multiple wires to convey inputs at the command end of the stylet to cause rotation and/or articulation of the distal end. As with the illustrated embodiments, an embodiment employing control wires may be able to articulate the distal end at least 90 degrees from its original, straightened position, and may be able to trace out a 360 degree arc as the stylet is rotated.

It should be further appreciated that the articulation end may comprise a coil-spring and an attachment member for coupling the coil spring to the control wires. The control wires may be distributed about the cross section of the clasping device and may then travel to the command end. As a control wire is tensioned by manipulation of the command end, the tensioned control wire will bend the articulation end of the tube in a direction of the tensioned control wire.

It should also be appreciated that, in still other aspects, a mechanical advantage system may be employed to amplify motion of the control member and create the desired tension in each wire. For example, the mechanical advantage system may comprise a basic ball-bushing joystick leverage system, the exemplary systems disclosed in U.S. Provisional Application No. 61/119,028, incorporated herein by reference, or any other conventional mechanical advantage command systems.

In should be appreciated that in still other aspects, the movement of joystick control members may generate an electronic signal. The signal will then control two servomotors, which will cause articulation of the stylet, for example, by applying tension to wires extending from the servomotors to a distal end of the stylet. The control member may be similar to joysticks found on most video game controllers, and is known as a dual-axis joystick. According to various aspects, the first and second servomotors may be oriented at 90 degrees relative to one another. First and second shafts may extend from the first and second servomotors, respectively. Each of the shafts may be attached to a respective pulley. Wires may be attached at each end of the flat portions of the pulleys. Opposite ends of the wires may be attached to the outer shaft at the distal end of the stylet in the same 90 degree orientation as they are arranged at the pulleys. In operation, movement of the control member may be translated into an electronic signal, and then passed to a battery pack and circuits where the signal will be processed. The processed signals may then be sent to the first and/or second servomotors. The signaled servomotor(s) will cause the corresponding pulley(s) to rotate up to at least 90 degrees clockwise and up to at least 90 degrees counterclockwise. For example, when the control member is moved left-to-right, the first servomotor rotates the pulley and causes the tip portion to move left-to-right. And when the control member is moved front-to-back, the second servomotor rotates the pulley and causes the tip portion to move front-to-back. As the control member is rotated in a 360 degree circle, the first and second servomotors both rotate and combine to make the tip of the stylet make a similar 360 degree circle.

The various embodiments of FIGS. 1-5 illustrate means by which one end of a relatively stiff tube can be articulated remotely from the other end. Articulation can be achieved within a full range of a half dome described by a radius. The high degree of articulation at the camera end may allow practitioners greater control over camera motion while searching for the trachea. A small video screen may contribute to a small, highly portable package. The aforementioned exemplary embodiments may allow medical practitioners to more easily and successfully perform the process of intubation in and out of the hospital.

It will be apparent to those skilled in the art that various modifications and variations can be made to the intubation stylets and the systems and methods for intubation of the present disclosure without departing from the scope of the invention. Other embodiments of the invention will be apparent to those skilled in the art from consideration of the specification and practice of the invention disclosed herein. It is intended that the specification and examples be considered as exemplary only.

What is claimed is:

1. An intubating stylet having a proximal end and a distal end, the intubating stylet comprising:
a housing at the proximal end of the stylet;
a tip portion at the distal end of the stylet, the tip portion having a distal gear at its proximal end;
an outer shaft extending from the housing to the tip portion, the outer shaft containing an inner shaft defining a hollow lumen, a distal end of the inner shaft having a proximal gear coupled with the distal gear of the tip portion such that rotation of the inner shaft causes articulation of the tip portion;
a drive member in the housing, the drive member being coupled to the inner shaft and configured to rotate the inner shaft; and
a control member coupled with the drive member, the control member being configured to receive a user input and to translate the input into operation of the drive member.

2. The intubating stylet of claim 1, wherein the drive member comprises a mechanical drive.

3. The intubating stylet of claim 2, wherein the drive member comprises a gear assembly.

4. The intubating stylet of claim 3, wherein the gear assembly comprises a first gear associated with the control member and a second gear associated with the inner shaft.

5. The intubating stylet of claim 4, wherein the first and second gears are mechanically coupled to one another such that a user input via the control member causes rotation of the inner shaft relative to the outer shaft and the housing via the gear assembly.

6. The intubating stylet of claim 5, wherein the first, second, proximal, and distal gears comprise bevel gears.

7. The intubating stylet of claim 6, wherein the control member comprises a dial configured to be operated by a user's thumb.

8. The intubating stylet of claim 7, further comprising:
a camera configured to capture images proximate the tip portion of the stylet; and
a display pivotally coupled to the housing, the display being configured to display the images captured by the camera.

9. The intubating stylet of claim 1, further comprising a second drive member coupled with the outer shaft and configured to rotate the outer shaft relative to the housing.

10. The intubating stylet of claim 9, further comprising a second control member coupled with the second drive member, the second control member being configured to receive a user input and to translate the input into operation of the second drive member.

11. The intubating stylet of claim 10, wherein the drive member and the second drive member each comprise a mechanical drive.

12. The intubating stylet of claim 11, wherein the drive member comprises a first gear assembly and the second drive member comprises a second gear assembly.

13. The intubating stylet of claim 12, wherein the first gear assembly comprises a first gear associated with the control member and a second gear associated with the inner shaft.

14. The intubating stylet of claim 13, wherein the first and second gears are mechanically coupled to one another such that a user input via the control member causes rotation of the inner shaft relative to the outer shaft and the housing via the first gear assembly.

15. The intubating stylet of claim 14, wherein the second gear assembly comprises a third gear associated with the second control member and a fourth gear associated with the outer shaft.

16. The intubating stylet of claim 15, wherein the third and fourth gears are mechanically coupled to one another such that a user input via the second control member causes rotation of the outer shaft relative to the housing via the second gear assembly

17. The intubating stylet of claim 16, wherein the first, second, third, further, proximal, and distal gears comprise bevel gears.

18. The intubating stylet of claim 17, wherein the control member and the second control member each comprise a dial configured to be operated by a user's thumb.

19. The intubating stylet of claim 18, further comprising:
a camera configured to capture images proximate the tip portion of the stylet; and
a display pivotally coupled to the housing, the display being configured to display the images captured by the camera.
